(19) United States
(12) Patent Application Publication
(10) Pub. No.: US 2015/0225370 A1
(43) Pub. Date: Aug. 13, 2015

(54) PROCESS FOR THE PREPARATION OF DABIGATRAN ETEXILATE OR PHARMACEUTICALLY ACCEPTABLE SALT THEREOF

(71) Applicant: RANBAXY LABORATORIES LIMITED, New Delhi, Delhi (IN)

(72) Inventors: Anandam Vempali, Nellore (IN); Sudhir Singh Sanwal, Kangra (IN); Balaguru Murugesan, Coimbatore (IN); Swargam Sathyanarayana, Gurgaon (IN); Rajesh Kumar Thaper, Jammu (IN); Mohan Prasad, Gurgaon (IN)

(21) Appl. No.: 14/430,324

(22) PCT Filed: Sep. 30, 2013
(86) PCT No.: PCT/IB2013/059017

§ 371 Date: Mar. 23, 2015

(30) Foreign Application Priority Data
Sep. 28, 2012 (IN) 3067/DEL/2012

(51) Int. Cl. C07D 401/12; C07C 303/32

(52) U.S. Cl. CPC C07D 401/12; C07C 303/32

(57) ABSTRACT

The present invention provides hydrobromide salt of dabigatran etexilate of formula (IV) and its process for the preparation. The present invention further provides crystalline Form I and crystalline Form II of hydrobromide salt of dabigatran etexilate and processes for their preparation. The present invention further relates to a process for the preparation of pharmaceutically ac ceptable salts, including methanesulfonate salt, of dabigatran etexilate using hydrobromide salt of dabigatran etexilate of the present invention.

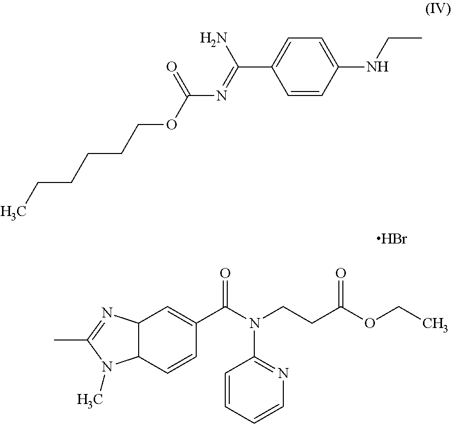

### FIELD OF THE INVENTION

The present invention provides hydrobromide salt of dabigatran etexilate and its process for the preparation. The present invention further provides crystalline Form I and crystalline Form II of hydrobromide salt of dabigatran etexilate and processes for their preparation. The present invention further relates to a process for the preparation of pharmaceutically acceptable salts, including methanesulfonate salt, of dabigatran etexilate using hydrobromide salt of dabigatran etexilate of the present invention.

### BACKGROUND OF THE INVENTION

The drug substance used in the commercial drug product formulation of Pradaxa® is the methanesulfonate salt of dabigatran etexilate, which is chemically described as β-Alanine, N-[[2-[[[4-[[[(hexyloxy)carbonyl]amino]iminomethyl]phenyl]amino]methyl]-1-methyl-1H-benzimidazol-5-yl]carbonyl]-N-2-pyridinyl-,ethyl ester, methanesulfonate salt of Formula I.

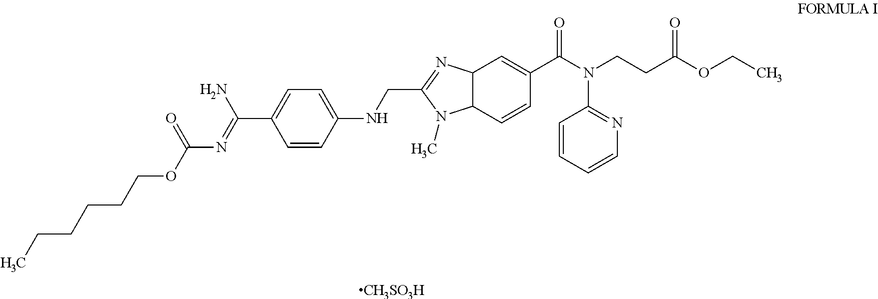 [SMILES: CCCCCCOC(=O)/N=C(\N)c1ccc(NCC2=NC3C=C(C(=O)N(CCC(=O)OCC)c4ccccn4)C=CC3N2C)cc1 ; "·CH3SO3H"]

Dabigatran etexilate of Formula II

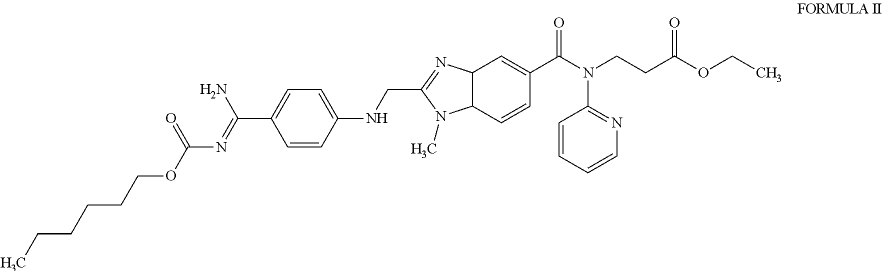 [SMILES: CCCCCCOC(=O)/N=C(\N)c1ccc(NCC2=NC3C=C(C(=O)N(CCC(=O)OCC)c4ccccn4)C=CC3N2C)cc1]

is a prodrug of dabigatran of Formula III

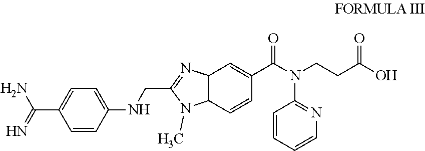 [SMILES: CN1C(CNc2ccc(C(=N)N)cc2)=NC2C=C(C(=O)N(CCC(=O)O)c3ccccn3)C=CC21]

which is a direct thrombin inhibitor. Dabigatran etexilate is indicated to reduce the risk of stroke and systemic embolism in patients with non-valvular atrial fibrillation. It may be used alone or in combination with other therapeutic agents.

Processes for the preparation of dabigatran etexilate or its different salts are described in U.S. Pat. No. 6,087,380; European Patent Publication No. EP 1870100 (equivalent to CA 2,476,054);; and PCT Publication Nos. WO 2006/114415 (equivalent to US 2006/0247278), WO 2008/043759, WO 2012/044595, WO 2012/027543, WO 2008/059029, WO 2011/110876, WO 2011/110478, and WO 2006/131491 (equivalent to US 2006/276513).

### SUMMARY OF THE INVENTION

The present invention provides the hydrobromide salt of dabigatran etexilate and its process for the preparation. The present invention further provides crystalline Form I and crystalline Form II of hydrobromide salt of dabigatran etexilate and processes for their preparation. The present invention further relates to a process for the preparation of pharmaceutically acceptable salts, including the methanesulfonate salt, of dabigatran etexilate using hydrobromide salt of dabigatran etexilate of the present invention.

### BRIEF DESCRIPTION OF THE DRAWINGS

FIG. 1 depicts the X-ray powder diffraction (XRPD) pattern of the crystalline Form I of hydrobromide salt of dabigatran etexilate obtained according to Example 1.

FIG. 1A provides the XRPD pattern of the crystalline Form I of hydrobromide salt of dabigatran etexilate depicted in FIG. 1.

FIG. 2 depicts the XRPD pattern of the crystalline Form II of hydrobromide salt of dabigatran etexilate obtained according to Example 2.

FIG. 2A provides the XRPD pattern of the crystalline Form II of hydrobromide salt of dabigatran etexilate depicted in FIG. 2.

### DETAILED DESCRIPTION OF THE INVENTION

A first aspect of the present invention provides the hydrobromide salt of dabigatran etexilate salt of Formula IV.

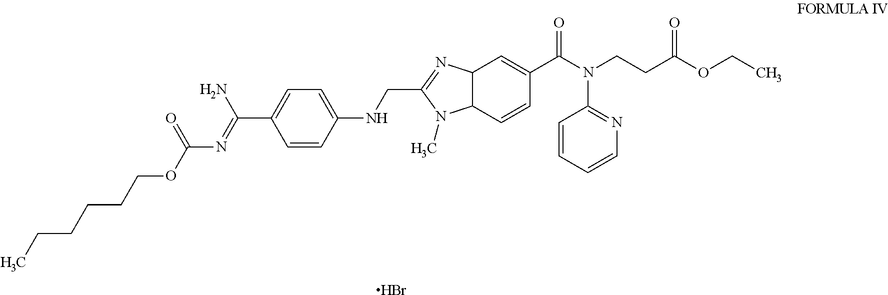 [SMILES: CCCCCCOC(=O)/N=C(\N)c1ccc(NCC2=NC3C=C(C(=O)N(CCC(=O)OCC)c4ccccn4)C=CC3N2C)cc1 ; Br]

A second aspect of the present invention provides a process for the preparation of the hydrobromide salt of dabigatran etexilate, wherein the process comprises:
a) contacting ethyl N-[(2-{[(4-carbamimidoylphenyl)amino]methyl}-1-methyl-3a,7a-dihydro-1H-benzimidazol-5-yl)carbonyl]-N-pyridin-2-yl-β-alaninate of Formula V

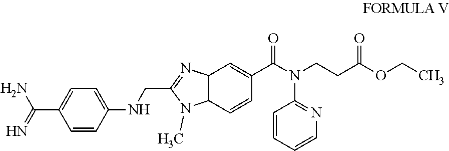 [SMILES: CCOC(=O)CCN(C(=O)C1=CC2N=C(CNc3ccc(C(=N)N)cc3)N(C)C2C=C1)c1ccccn1]

or its salt with n-hexyl chloroformate;
b) treating the reaction mixture obtained in step a) with hydrobromic acid; and
c) isolating hydrobromide salt of dabigatran etexilate of compound of Formula IV from the mixture thereof.

The ethyl N-[(2-{[(4-carbamimidoylphenyl)amino]methyl}-1-methyl-3a,7a-dihydro-1H-benzimidazol-5-yl)carbonyl]-N-pyridin-2-ylβ-alaninate of Formula V, or its salt may be prepared according to methods provided in literature, for example, U.S. Pat. No. 6,087,380.

The salts of compound of ethyl N-[(2-{[(4-carbamimidoylphenyl)amino]methyl}-1-methyl-3a,7a-dihydro-1H-benzimidazol-5-yOcarbonyl]-N-pyridin-2-ylβ-alaninate of Formula V may be selected from hydrochloride, hydrobromide, or acetate salt. Preferably, the salt of compound of Formula V is an acetate salt.

The compound of Formula V or its salt is contacted with n-hexyl chloroformate in the presence of a solvent selected from the group consisting of water, ethers, halogenated hydrocarbons, esters, or mixtures thereof. The ether solvent may be selected from the group comprising tetrahydrofuran, diisopropyl ether, or methyl t-butyl ether. The halogenated hydrocarbon solvent may be dichloromethane. The ester solvent may be ethyl acetate. Preferably, the solvent is tetrahydrofuran, either alone or in combination with water. The n-hexyl chloroformate may be used either as a solid or in solution form with tetrahydrofuran.

The compound of Formula V or its salt is contacted with the n-hexyl chloroformate in the presence of an organic or inorganic base. The organic base may be selected from the group comprising ethylamine or diisopropyl ethyl amine The inorganic base may be selected from the group comprising sodium carbonate or potassium carbonate. Preferably, the base is potassium carbonate.

The compound of Formula V or its salt is contacted with the n-hexyl chloroformate at a temperature of about 10° C. to about 40° C., for example, about 15° C. to about 25° C. The compound of Formula V or its salt may be contacted with n-hexyl chloroformate for about 3 hours to about 6 hours, for example, about 4 hours to about 6 hours.

The reaction mixture may be subjected to carbon treatment. The reaction mixture may optionally be treated with butylated hydroxytoluene. The solvent may be recovered from the reaction mixture and the reaction mixture used as such for the next step.

The reaction mixture obtained in step a) is treated with hydrobromic acid in the presence of a solvent selected from the group consisting of ketones, esters, alcohols, or mixtures thereof. The ketone solvent may be selected from the group comprising acetone, methyl butyl ketone, or methyl isopropyl ketone. The ester solvent may be selected from the group comprising ethyl acetate, isopropyl acetate, or butyl acetate. The alcohol solvent may be selected from the group comprising ethanol, methanol, n-propanol, or butanol. Preferably, the solvent is acetone. The hydrobromic acid may be used as a solid or in solution form with acetone.

The reaction mixture obtained in step a) is treated with hydrobromic acid at a temperature of about 10° C. to about 40° C., for example, about 15° C. to about 25° C. The reaction mixture obtained in step a) is treated with hydrobromic acid for about 3 hours to about 6 hours, for example, about 4 hours to about 6 hours.

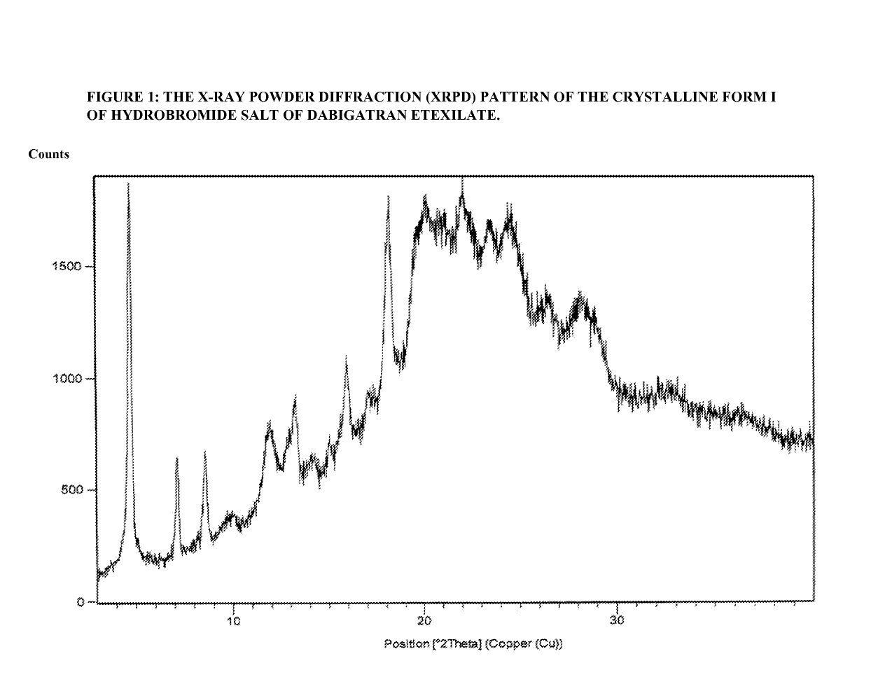

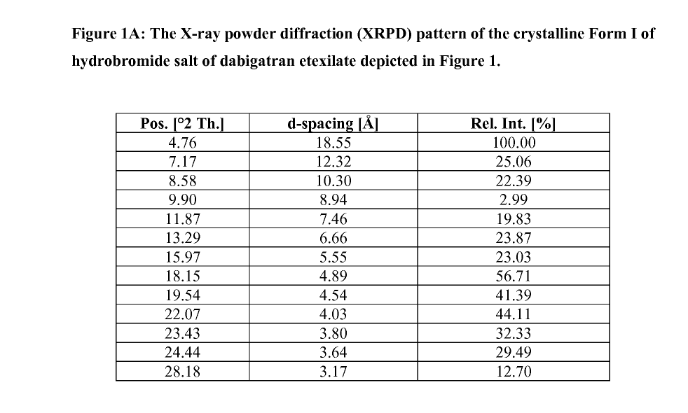

The hydrobromide salt of dabigatran etexilate may be isolated by filtration, decantation, evaporation, distillation or a combination thereof. The hydrobromide salt of dabigatran etexilate has substantially the same X-ray powder diffraction (XRPD) pattern as depicted in FIG. 1, and is referred to herein as crystalline Form I of the hydrobromide salt of dabigatran etexilate.

A third aspect of the present invention provides crystalline Form I of the hydrobromide salt of dabigatran etexilate.

The crystalline Form I of the hydrobromide salt of dabigatran etexilate has substantially the same XRPD pattern as depicted in FIG. 1. The crystalline Form I of the hydrobromide salt of dabigatran etexilate salt of Formula IV is characterized by an XRPD pattern having interplanar spacing (d) values substantially at 18.55, 4.89, 4.54, 4.03, and 3.80 Å. The crystalline Form I of the hydrobromide salt of dabigatran etexilate salt of Formula IV is further characterized by an XRPD pattern having interplanar spacing (d) values substantially at 18.55, 12.32, 10.30, 8.94, 7.46, 6.66, 5.55, 4.89, 4.54, 4.03, 3.80, 3.64, and 3.17 Å.

A fourth aspect of the present invention provides a process for the purification of the hydrobromide salt of dabigatran etexilate, wherein the process comprises:
a) treating the hydrobromide salt of dabigatran etexilate of Formula IV with an alcohol solvent; and
b) isolating the purified hydrobromide salt of dabigatran etexilate of Formula IV from the mixture thereof.

The alcohol solvent used for purification may be selected from the group comprising methanol, ethanol, isopropanol, n-propanol, or mixtures thereof. Preferably, the alcohol solvent is ethanol. The hydrobromide salt of dabigatran etexilate is treated with an alcohol solvent at a temperature of about 10° C. to about 70° C., for example, about 20° C. to about 60° C. The hydrobromide salt of dabigatran etexilate is treated with an alcohol solvent for about 2 hours to about 6 hours, for example, about 3 hours to about 4 hours.

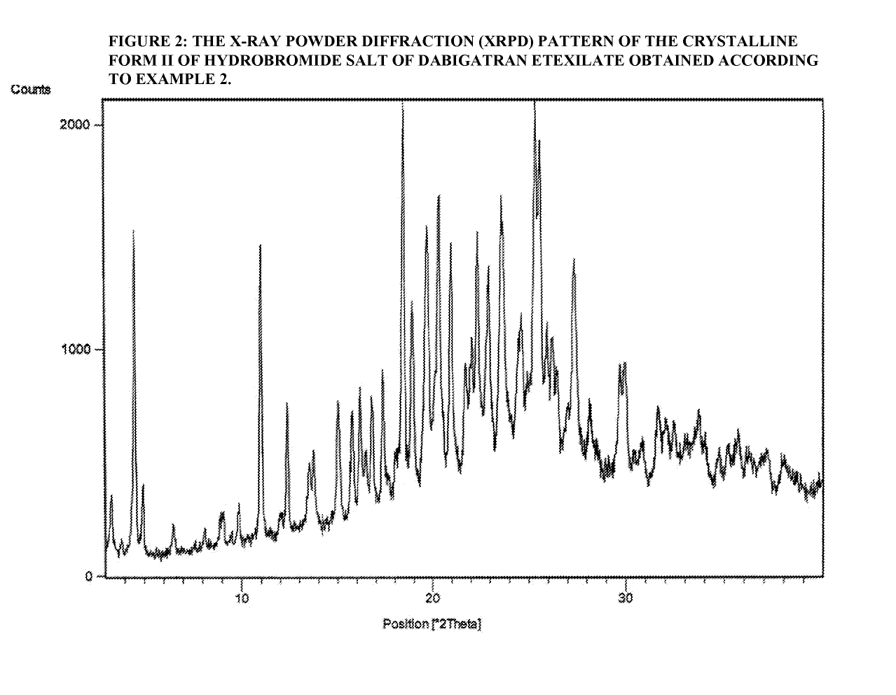

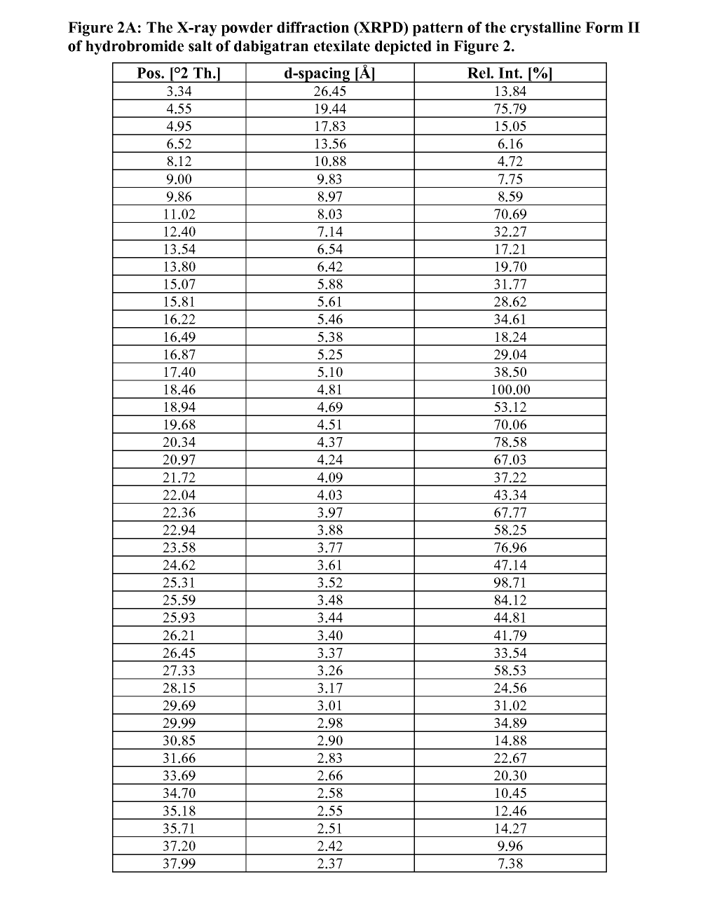

The purified hydrobromide salt of dabigatran etexilate may be isolated by filtration, decantation, evaporation, distillation, or combinations thereof. The purified hydrobromide salt of dabigatran etexilate has substantially the same XRPD pattern as depicted in FIG. 2, and is referred to herein as crystalline Form II of hydrobromide salt of dabigatran etexilate.

A fifth aspect of the present invention provides crystalline Form II of hydrobromide salt of dabigatran etexilate.

The crystalline Form II of hydrobromide salt of dabigatran etexilate has substantially the same XRPD pattern as depicted in FIG. 2. The crystalline Form II of hydrobromide salt of dabigatran etexilate is characterized by an XRPD pattern having interplanar spacing (d) values substantially at 19.44, 8.03, 4.81, 4.69, 4.51, 4.37, 4.24, 3.97, 3.77, and 3.52 Å. The crystalline Form II of the hydrobromide salt of dabigatran etexilate is further characterized by an XRPD pattern having interplanar spacing (d) values substantially at 26.45, 19.44, 17.83, 13.56, 10.88, 9.83, 8.97, 8.03, 7.14, 6.54, 6.42, 5.88, 5.61, 5.46, 5.38, 5.25, 5.10, 4.81, 4.69, 4.51, 4.37, 4.24, 4.09, 4.03, 3.97, 3.88, 3.77, 3.61, 3.52, 3.48, 3.44, 3.40, 3.37, 3.26, 3.17, 3.01, 2.98, 2.90, 2.83, 2.66, 2.58, 2.55, 2.51, 2.42, and 2.37 Å.

A sixth aspect of the present invention provides a process for the preparation of the methanesulfonate salt of dabigatran etexilate, wherein the process comprises:
a) treating the hydrobromide salt of dabigatran etexilate of Formula IV with methanesulfonic acid; and
b) isolating the methanesulfonate salt of dabigatran etexilate from the mixture thereof.

The hydrobromide salt of dabigatran etexilate of Formula IV may be treated with a suitable acid to prepare the pharmaceutically acceptable salts of dabigatran etexilate. Pharmaceutically acceptable salts of dabigatran etexilate may be, for example, the methanesulfonate salt of dabigatran etexilate. The hydrobromide salt of dabigatran etexilate of Formula IV is treated with a solvent and a base before treating with methanesulfonic acid. The solvent may be selected from the group consisting halogenated hydrocarbons, esters, ketones, alcohols, or mixtures thereof. The halogenated hydrocarbon may be dichloromethane. The ester solvent may be selected from the group comprising ethyl acetate, isopropyl acetate, or butyl acetate. The ketone solvent may be selected from the group comprising acetone, methyl butyl ketone, or methyl isopropyl ketone. The alcohol solvent may be selected from the group comprising ethanol, methanol, n-propanol, or butanol. Preferably, the solvent is dichloromethane, ethyl acetate, or a mixture thereof.

The base may be an inorganic base or an organic base. The inorganic base may be, for example, sodium carbonate or potassium carbonate. The organic base may be, for example, ethyl amine, isopropyl amine, or diisopropylethyl amine Preferably, the base is sodium carbonate or potassium carbonate. The hydrobromide salt of dabigatran etexilate of Formula IV is treated with a solvent and a base at a temperature of about 10° C. to about 80° C., for example, about 20° C. to about 60° C. The hydrobromide salt of dabigatran etexilate of Formula IV is treated with a solvent and a base for about 30 minutes to about 3 hours, for example, about 1 hour to about 2 hours.

The hydrobromide salt of dabigatran etexilate of Formula IV may be treated with methanesulfonic acid in the presence of a solvent selected from the group consisting of ketones, esters, alcohols, or mixtures thereof. The ketone solvent may be selected from the group comprising acetone, methyl butyl ketone, or methyl isopropyl ketone. The ester solvent may be selected from the group comprising ethyl acetate, isopropyl acetate, or butyl acetate. The alcohol solvent may be selected from the group comprising ethanol, methanol, n-propanol, or butanol. Preferably, the solvent is ethyl acetate. The methanesulfonic acid may be used as a solid or in the solution form with ethyl acetate.

The hydrobromide salt of dabigatran etexilate is treated with methanesulfonic acid at a temperature of about 10° C. to about 60° C., for example, about 20° C. to about 50° C. The hydrobromide salt of dabigatran etexilate is treated with methanesulfonic acid for about 3 hours to about 6 hours, for example, about 4 hours to about 6 hours.

The methanesulfonate salt of dabigatran etexilate may be isolated by filtration, decantation, evaporation, distillation, or combinations thereof. The methanesulfonate salt of dabigatran etexilate prepared by the present invention may be characterized by XRPD pattern.

The XRPD of the samples were determined by using a PANalytical X'Pert PRO X-Ray Powder Diffractometer in the range 3-40 degree 2 theta and under tube voltage and current of 45 Kv and 40 mA respectively. Copper radiation of wavelength 1.54 angstrom and X'Celerator detector was used.

While the present invention has been described in terms of its specific embodiments, certain modifications and equivalents will be apparent to those skilled in the art and are intended to be included within the scope of the present invention.

### EXAMPLES

### Example 1

### Preparation of Dabigatran Etexilate Hydrobromide Salt

The acetate salt of ethyl N-[(2-{[(4-carbamimidoylphenyl)amino]methyl}-1-methyl-3a,7a-dihydro-1H-benzimidazol-5-yOcarbonyl]-N-pyridin-2-yl-β-alaninate (50 g) was added to tetrahydrofuran (750 mL) and deionized water (250 mL) and the reaction mixture was stirred for 20 minutes. Potassium carbonate (37.08 g) was added to the reaction mixture and the reaction mixture was stirred for 30 minutes. A solution of n-hexyl chloroformate (16.19 g) dissolved in tetrahydrofuran (250 mL) was added to the reaction mixture at 18° C. to 20° C. The reaction mixture was stirred for 2 hours at 20° C. to 22° C. The tetrahydrofuran layer was collected. Potassium carbonate (40 g) was added to the reaction mixture, and the reaction mixture was stirred for 30 minutes.

The layers obtained were separated and the tetrahydrofuran layer was used further. Carbon (5 g) was added to the reaction mixture and stirred for 20 minutes. The reaction mixture was filtered through celite. The tetrahydrofuran layer was collected and butylated hydroxytoluene (BHT) (0.5 g) was added to the reaction mixture. The solvents were recovered under vacuum. Acetone (150 mL) was added to the reaction mixture and stirred for 20 minutes. The acetone was recovered under vacuum. The solid obtained was dissolved in acetone (392 mL). A solution of 45% hydrobromic acid (15.24 g) in acetone (56 mL) was added to the reaction mixture at 18° C. to 20° C. The reaction mixture was stirred at 20° C. to 22° C. for 2 hours, filtered, and dried under suction. The reaction mixture was further dried under vacuum at 55° C. for 15 hours to obtain the title compound having XRPD data as depicted in FIG. 1.

### Yield: 42 g

### Example 2

### Purification of Dabigatran Etexilate Hydrobromide Salt

Dabigatran etexilate hydrobromide salt (40 g) obtained in Example 1 was dissolved in ethanol (280 mL) at 55° C. for 15 minutes to 20 minutes. The reaction mixture was cooled to 10° C. to 15° C. for 20 minutes. The reaction mixture was stirred for 2 hours at 20° C., filtered and dried under suction. The reaction mixture was washed with ethanol (50 mL), and then dried under vacuum at 55° C. for 15 hours to obtain the title compound having XRPD data as depicted in FIG. 2.

### Yield: 42 g

(M+H)⁺: m/z=628 ¹H NMR (400 MHz, CDCl₃): δ0.86-0.89 (t,3H), 1.10-1.13 (t,3H), 1.29-1.30 (m,6H), 1.65-1.67 (m,2H), 2.66-2.69 (t,2H), 3.78 (s,3H), 3.94-3.99 (t,2H), 4.20-4.27 (m,4H), 4.69-4.70 (d,2H), 6.86-6.89 (m,3H), 6.91-7.17 (m,2H), 7.41-7.43 (m,2H), 7.47 (t,1H), 7.55-7.66 (dt,3H), 8.37-8.39 (dd,1H), 10.0 (s,1H), 10.65 (bs,1H), 11.90 (bs,1H)

### Example 3

### Preparation of Dabigatran Etexilate Methanesulfonate Salt

Dabigatran etexilate hydrobromide salt (35 g) was dissolved in dichloromethane (350 mL) at 25° C. A 5% aqueous sodium carbonate solution (210 mL) was added to the reaction mixture and stirred for 10 minutes. The dichloromethane layer was separated and the dichloromethane was recovered under vacuum. Ethyl acetate (550 mL) was added to the reaction mixture and stirred for 10 minutes. Methane sulphonic acid solution (3.99 g methane sulphonic acid dissolved in 55 mL ethyl acetate) was added to the reaction mixture drop-wise at 20° C. to 25° C. The reaction mixture was stirred at 20° C. to 25° C. for 2 hours. The reaction mixture was filtered under vacuum and washed with ethyl acetate (27 mL). The solid obtained was dried under vacuum at 55° C. for 14 hours to 15 hours to obtain the title compound.

Yield: 29.75 g

1. A hydrobromide salt of dabigatran etexilate salt of Formula IV.

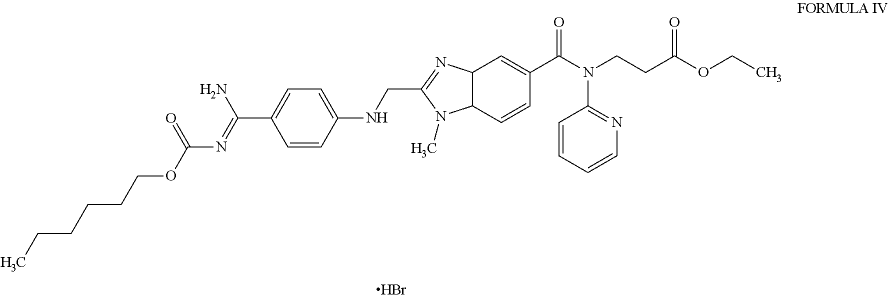 [SMILES: CCCCCCOC(=O)/N=C(\N)c1ccc(NCC2=NC3C=C(C(=O)N(CCC(=O)OCC)c4ccccn4)C=CC3N2C)cc1 ; Br]

2. A process for the preparation of a hydrobromide salt of dabigatran etexilate of Formula IV, wherein the process comprises:
a) contacting ethyl N-[(2-{[(4-carbamimidoylphenyl)amino]methyl}-1-methyl-3a,7a-dihydro-1H-benzimidazol-5-yl)carbonyl]-N-pyridin-2-yl-β-alaninate of Formula V or its salt with n-hexyl chloroformate;
b) treating the reaction mixture obtained in step a) with hydrobromic acid; and
c) isolating hydrobromide salt of dabigatran etexilate of Formula IV from the mixture thereof.

3. The process according to claim 2, wherein the salt of compound of ethyl N-[(2-{[(4-carbamimidoylphenyl)amino]methyl}-1-methyl-3a,7a-dihydro-1H-benzimidazol-5-yl)carbonyl]-N-pyridin-2-yl-β-alaninate of Formula V is selected from hydrochloride, hydrobromide, or acetate salts.

4. (canceled)

5. The process according to claim 2, wherein the compound of Formula V or its salt is contacted with n-hexyl chloroformate in the presence of a solvent selected from the group consisting of water, ether, halogenated hydrocarbon, ester, or mixtures thereof.

6. The process according to claim 5, wherein the solvent is tetrahydrofuran or tetrahydrofuran in combination with water.

7. The process according to claim 2, wherein the compound of Formula V or its salt is contacted with n-hexyl chloroformate in the presence of a base.

8. The process according to claim 7, wherein the base is potassium carbonate.

9. The process according to claim 2, wherein the reaction mixture obtained in step a) is treated with hydrobromic acid in the presence of a solvent selected from the group consisting of ketones, esters, alcohols, or mixtures thereof.

10. The process according to claim 9, wherein the ketone solvent is acetone.

11-14. (canceled)

15. A process for the purification of a hydrobromide salt of dabigatran etexilate, wherein the process comprises:
a) treating hydrobromide salt of dabigatran etexilate of Formula IV with an alcohol solvent; and
b) isolating purified hydrobromide salt of dabigatran etexilate of Formula IV from the mixture thereof.

16. The process according to claim 15, wherein the alcohol solvent is ethanol.

17-20. (canceled)

21. A process for the preparation of a methane sulfonate salt of dabigatran etexilate, wherein the process comprises:
a) treating hydrobromide salt of dabigatran etexilate of Formula IV with methanesulfonic acid; and
b) isolating the methanesulfonate salt of dabigatran etexilate from the mixture Thereof.

22. The process according to claim 21, wherein the hydrobromide salt of dabigatran etexilate of Formula IV is treated with a solvent and a base before treating with methanesulfonic acid.

23. The process according to claim 21, wherein the hydrobromide salt of dabigatran etexilate of Formula IV is treated with methanesulfonic acid in the presence of a solvent selected from the group consisting of ketones, esters, alcohols, or mixtures thereof.

24. The process according to claim 23, wherein the ester solvent is ethyl acetate.
